(19) United States
(12) Patent Application Publication
(10) Pub. No.: US 2025/0019748 A1
(43) Pub. Date: Jan. 16, 2025

(54) ATTACHING NUCLEIC ACIDS TO A PLATINUM ELECTRODE

(71) Applicants: UNIVERSITE PARIS-SACLAY, GIF-SUR-YVETTE (FR); CENTRE NATIONAL DE LA RECHERCHE SCIENTIFIQUE (CNRS), PARIS (FR)

(72) Inventors: Jean GAMBY, SACLAY (FR); Jeremy LE GALL, PARIS (FR); Djamila KECHKECHE, AULNAY SOUS BOIS (FR); Claire POUJOULY, Gif sur Yvette (FR)

(73) Assignees: UNIVERSITE PARIS-SACLAY, GIF-SUR-YVETTE (FR); CENTRE NATIONAL DE LA RECHERCHE SCIENTIFIQUE (CNRS), PARIS (FR)

(21) Appl. No.: 18/711,813

(22) PCT Filed: Nov. 30, 2022
(86) PCT No.: PCT/FR2022/052201

§ 371 Date: May 20, 2024

(30) Foreign Application Priority Data
Nov. 30, 2021 (FR) FR2112764

(51) Int. Cl. C12Q 1/6834; C12Q 1/6806

(52) U.S. Cl. CPC C12Q 1/6834; C12Q 1/6806

(57) ABSTRACT

The invention relates to a method for attaching nucleic acids to a platinum electrode, comprising the following steps: —a step (S1) of attaching an ethylenediamine molecule to the electrode, step (S1) comprising electro-oxidation of a primary amine from the ethylenediamine molecule by cyclic voltammetry, —a step (S2) of attaching a sulfo SMCC molecule of 4-(N-maleimidomethyl)cyclohexane-1-carboxylic acid 3-sulfo-N-hydroxysuccinimide ester to the ethylenediamine molecule, —a step (S3) of attaching a nucleic acid to the sulfo SMCC molecule, the nucleic acid having been modified beforehand to comprise a thiol function, the electrode being in contact with an aqueous solution, i.e. with a solution in which the solvent is water, during steps (S1), (S2) and (S3).

### FIELD OF THE INVENTION

The invention relates to attaching nucleic acids to a platinum electrode, and in particular to attaching nucleic acids to a platinum micro-electrode in an aqueous solution.

The invention also relates to a platinum electrode thus functionalized and the use of such an electrode for the detection of a target nucleic acid.

### STATE OF THE ART

Gene detection by electrochemical techniques is known. These techniques generally use a gold electrode to which a self-assembled monolayer (also known as SAM) is attached. The attachment of this monolayer conventionally uses a thiol function because it comprises a sulfur atom which has a strong affinity with the gold of the electrode. It is known that the stability of such self-assembled monolayers is not satisfactory due to chemical desorption processes. This is mainly due to the weak nature of the gold-sulfur “Au—S” bond for which the binding energy-40 kcal/mol is very low compared to a stronger covalent carbon-carbon “C—C” type bond around 85 kcal/mol. Additionally, the Au—S bond can be affected by its susceptibility to oxidation (change in degree of oxidation) or when the gold electrode is polarized. Moreover, the gold oxidation process is quite rapid in aqueous solutions, which tends to reduce the stability of such an electrode used in an aqueous medium.

There is therefore a need for an electrochemical detection device having better electrode stability.

### DISCLOSURE OF THE INVENTION

A purpose of the invention is to propose an electrochemical detection device having better stability of the electrode compared to the prior art.

The purpose is achieved in the context of the present invention thanks to a method for attaching nucleic acid to a platinum electrode comprising the following steps:
a step (S1) of attaching an ethylenediamine molecule to the electrode, step (S1) comprising electro-oxidation of a primary amine from the ethylenediamine molecule by cyclic voltammetry,
a step (S2) of attaching a sulfo SMCC molecule of 4-(N-Maleimidomethyl) cyclohexane-1-carboxylic acid 3-sulfo-N-hydroxysuccinimide ester to the ethylenediamine molecule,
a step (S3) of attaching a nucleic acid to the sulfo SMCC molecule, the nucleic acid having been modified beforehand to comprise a thiol function, the electrode being in contact with an aqueous solution, that is to say with a solution in which the solvent is water, during steps (S1), (S2) and (S3).

This method allows to functionalize a platinum electrode, that is to say in an electrode in a material which has greater electrochemical stability than gold. An electrochemical detection device which is based on such an electrode solves the problem mentioned above.

Such a method is advantageously and optionally supplemented by the following different characteristics taken alone or in combination:
the aqueous solution is a physiological solution;
the electrode is a micro-electrode placed at least partly in a micro-fluidic channel;
after step (S3) a stabilization step (S4) during which the electrode is placed in physiological solution for a duration comprised between 20 minutes and 40 minutes, the solution having a NaCl concentration comprised between 0.4 molar and 0.6 molar.

The invention also relates to a platinum electrode for the detection of a target nucleic acid comprising an ethylenediamine molecule attached to the electrode, a SMCC sulfo molecule of 4-(N-Maleimidomethyl)cyclohexane-1-carboxylic acid 3-sulfo-N-hydroxysuccinimide ester being attached to the ethylenediamine molecule by electro-oxidation of a primary amine from the ethylenediamine molecule by cyclic voltammetry, a probe nucleic acid being attached to the sulfo SMCC molecule, the probe nucleic acid comprising a thiol function, the probe nucleic acid being complementary to the target nucleic acid.

The invention further relates to a device for detecting a target nucleic acid comprising a platinum electrode as mentioned above, a counter electrode and an electrical measurement system electrically connected to the platinum electrode and to the counter electrode.

Such a device is advantageously and optionally supplemented by a micro-fluidic channel, the platinum electrode being a micro-electrode, the platinum electrode and the counter electrode being placed at least partly in the micro-fluidic channel.

The invention also relates to a system for detecting target nucleic acids comprising a plurality of devices as presented above, the system comprising an inlet configured to receive a solution to be analyzed, the inlet being connected to each platinum electrode of each device.

Such a system may advantageously and optionally be such that two of the plurality of devices are configured to detect two different target nucleic acids.

Finally, the invention relates to a method for detecting a target nucleic acid in a solution to be analyzed, the method comprising the following steps:
a step (E1) of providing a working electrode, the working electrode being a platinum electrode as presented above,
a step (E2) of providing a counter electrode,
a step (E3) of electrically energizing the working electrode and the counter electrode so as to maintain a working voltage between them,
a step (E4) of bringing the working electrode and the counter electrode into contact with the solution to be analyzed,
a step (E5) of determining a measured intensity of an electric current between the working electrode and the counter electrode,
a step (E6) of determining the presence of a target nucleic acid in the solution to be analyzed based on the measured intensity.

This method can advantageously and optionally be by:
a calibration step comprising the following sub-steps:
   a first sub-step (SE1) of supplying two reference solutions having different concentrations of target nucleic acid,
   a second sub-step (SE2) of measuring for each reference solution a reference intensity between the working electrode and the counter electrode, the electrodes being brought into contact with the reference solution, the electrodes being electrically energized so as to maintain the working voltage between them,
   a third sub-step (SE3) of determining a correspondence of current intensities between the working electrode and the counter electrode accordingly and of target nucleic acid concentrations in a solution to be analyzed, the method comprising a step of determining a target nucleic acid concentration in the solution to be analyzed from the measured intensity using the correspondence;
the target nucleic acid is a nucleic acid fragment of a pathogen.

### DESCRIPTION OF THE FIGURES

Other characteristics and advantages of the invention will emerge from the description which follows, which is purely illustrative and not limiting, and must be read with reference to the appended drawings in which:

FIGS. 1 to 3 are schematic representations of steps of a method for attaching a nucleic acid to an electrode according to one embodiment of the invention;

FIG. 4 is a schematic representation of a hybridization reaction of a target nucleic acid;

FIG. 5 is a schematic representation of electrochemical measurements for the detection of a target nucleic acid;

FIG. 6 is a schematic representation of a nucleic acid detection apparatus;

FIG. 7 is a detail of the apparatus shown in FIG. 6;

FIGS. 8 to 9 are schematic representations of electrochemical measurements for the detection of a target nucleic acid;

FIG. 10 is a schematic representation of a target nucleic acid detection system according to one embodiment of the invention.

### DETAILED DESCRIPTION OF THE INVENTION

### Method for Attaching Nucleic Acid to a Platinum Electrode

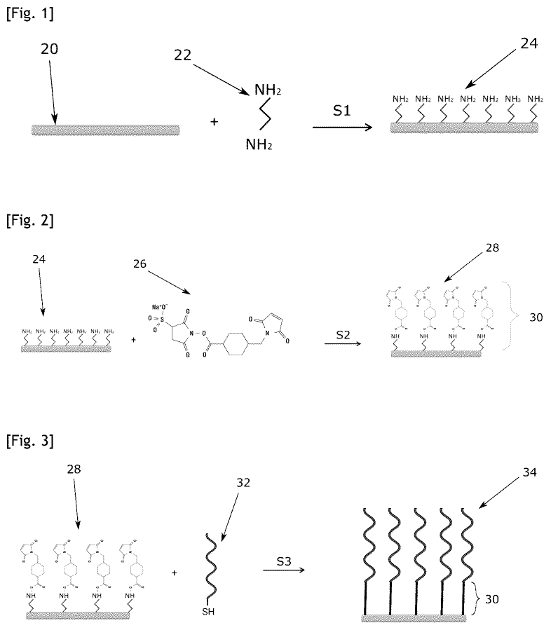

A method according to one embodiment of the invention for attaching a nucleic acid to a platinum electrode is presented in connection with FIGS. 1, 2 and 3. A platinum electrode 20 is initially provided. It is also referred to as a “working electrode” in the text of the description. In an optional preliminary step, this electrode can be characterized to evaluate its surface state and its electrochemical properties.

To this end, two measurements can be implemented in particular: a cyclic voltammetry measurement and a chronoamperometry measurement.

For these two measurements, the platinum electrode 20 is placed close to a counter electrode, and a liquid comprising Iron (III) ions, such as potassium ferricyanide, is brought into contact with the platinum electrode 20 and with the counter electrode. For example, a solution of potassium ferricyanide in an aqueous solution can be used. The solution comprising potassium ferricyanide also comprises ferrocyanide ions Fe(II), the Ferricyanide/Ferrocyanide mixture may be equimolar (1/1) or not. The concentration of potassium ferricyanide is chosen greater than or equal to 3 millimoles/liter and less than or equal to 30 millimoles/liter, for example equal to 20 millimoles/liter. The solution comprising Iron (III) ions can flow over the electrodes under a chosen flow greater than or equal to 0.1 μL/second and less than or equal to 1 μL/second, preferably equal to 0.5 L/second.

An aqueous solution is defined as a solution in which the solvent is water, that is to say whose majority component is water. A salt whose ions ensure ionic conductivity are generally dissolved in this solution. This assembly (solution+salt) is generally called supporting electrolyte.

An electric voltage is applied between the platinum working electrode 20 and the counter electrode and an electric current is measured between them.

The cyclic voltammetry involves scanning the voltage from −200 millivolts to +200 millivolts with a scan rate of 10 millivolts/second and measuring the current during the scan.

Chronoamperometry consists of keeping the voltage constant, for example at −200 mVolts, and measuring the electric current between the electrodes during a monitoring period, lasting for example 2 minutes.

It should be noted that an electrochemical impedance spectroscopy (EIS) measurement can be carried out in potentiostatic mode (also known as potentiostatic electrochemical impedance spectroscopy” abbreviated as PEIS) or in galvanostatic mode (also known as “Galvanostatic electrochemical impedance spectroscopy” abbreviated as GEIS). Technically, the PEIS measurement is implemented by scanning frequencies from 1.0 MHz to 100 mHz, to evaluate the electrochemical properties of the electrode. Thereupon, a zero potential difference is applied continuously between the platinum electrode and the counter electrode, as well as an alternating voltage of 10 millivolts, an alternating voltage which is frequency-scanned from 1.0 MHz to 100 mHz.

In a step S1, shown in FIG. 1, a molecule of ethylenediamine (term abbreviated below as EDA) 22 is attached to the electrode 20. An aqueous solution having a concentration of EDA is brought into contact with the platinum electrode 20. In particular the aqueous solution can be a physiological solution.

A physiological solution is defined as an aqueous solution having a sodium chloride concentration greater than or equal to 0.4 mole/liter and less than or equal to 0.6 mole/liter, for example a concentration of 0.5 mole/liter.

The EDA concentration may be greater than or equal to one millimole/liter and less than or equal to 10 millimoles/liter, for example a concentration of 2 millimoles/liter.

During attachment, a primary amine NH2 of the EDA molecule reacts with platinum, to form a Platinum+EDA product 24.

The attachment reaction comprises an electro-oxidation of a primary amine from the EDA molecule, which electro-oxidation is monitored by cyclic voltammetry. The voltage can be swept in several cycles, for example ten cycles, from 0 Volts to +1200 mVolts.

Electro-oxidation consists of covalently grafting a layer of organic molecules thanks to an electrochemical reaction of oxidation or reduction of a particular chemical group on the surface of an electrode. Here, electro-oxidation of the primary amine on the platinum electrode allows to carry out electrografting of ethylenediamine. A covalent bond is thus created between a nitrogen of the ethylenediamine and the surface of the platinum electrode.

Several EDA molecules can be attached to the electrode 20 during step S1, so that a self-assembled monolayer of EDA molecules is formed on the surface of the electrode.

At the end of step S1, a step of characterizing the Platinum+EDA electrode 24 can be implemented, using one of the measurements previously described: cyclic voltammetry and/or chronoamperometry.

In a step S2, shown in FIG. 2, a sulfo SMCC molecule of 4-(N-Maleimidomethyl)cyclohexane-1-carboxylic acid 3-sulfo-N-hydroxysuccinimide ester 26 is attached to the molecule of ethylenediamine 22 previously attached to the platinum electrode 20.

More precisely, the sulfo SMCC molecule 26 is attached to a distal end of the EDA molecule 22, this distal end being opposite a proximal end of the EDA molecule which is attached to the platinum electrode 20.

The sulfo SMCC molecule 26 has an N-hydroxysuccinimide function (commonly abbreviated to “NHS”) which reacts with a second amine of the EDA molecule, so as to form a product Platinum+EDA+sulfo SMCC 28. The second amine of the molecule EDA constitutes the distal end.

For this step S2, an aqueous solution having a concentration of sulfo SMCC molecule is brought into contact with the Platinum+EDA electrode 24. In particular the aqueous solution can be a physiological solution.

The concentration of SMCC sulfo molecule may be greater than or equal to one millimole/liter and less than or equal to 100 millimoles/liter, for example a concentration of ten millimoles/liter.

This attachment reaction can in particular be carried out at pH greater than or equal to 7.5 and less than or equal to 9 and statically over a period greater than or equal to 45 minutes and less than or equal to 1 hour 15 minutes, preferably equal to 1 hour. A static reaction here means that a quantity of solution comprising sulfo SMCC molecules, a single block and fixed quantity, is brought into contact with the platinum electrode. In particular, there is no flow of solution flowing over the platinum electrode.

During step S2, several sulfo SMCC molecules can each be attached to an EDA molecule attached to the electrode 20. The self-assembled monolayer of EDA molecules previously mentioned is then completed with the sulfo SMCC molecules.

The EDA+sulfo SMCC assembly attached above the electrode is referred to as a linker referenced 30 in FIG. 2.

At the end of step S2, a step of characterizing the Platinum+EDA+sulfo SMCC 28 electrode can be implemented, using one of the measurements previously described: cyclic voltammetry and/or chronoamperometry and/or PEIS.

In a step S3, shown in FIG. 3, a nucleic acid 32 is attached to the sulfo SMCC molecule 26 previously attached to the platinum electrode 20 via the EDA molecule 22.

More specifically, the nucleic acid 32 is attached to a distal end of the sulfo SMCC molecule 26 opposite a proximal end of the sulfo SMCC molecule 26 which is attached to the distal end of the EDA molecule 22.

The nucleic acid 32 is previously modified to comprise at one of its ends a thiol function, that is to say a —SH function comprising a sulfur atom S and a hydrogen atom H. This is thiol function which allows the nucleic acid to be attached to the sulfo SMCC molecule 26, and more precisely to a maleimide function of the sulfo SMCC molecule 26.

For this step S3, an aqueous solution having a nucleic acid concentration is brought into contact with the Platinum+EDA+sulfo SMCC electrode 28. In particular the aqueous solution can be a physiological solution.

The concentration of nucleic acid molecule may be greater than or equal to 0.1 micromole/liter and less than or equal to 10 micromoles/liter, for example a concentration of 1 micromole/liter.

This attachment reaction of step S3 can in particular be carried out statically over a period greater than or equal to 1 hour 45 minutes and less than or equal to 2 hours 15 minutes, preferably equal to 2 hours.

Once the nucleic acid 32 is attached to the “linker” 30, a Platinum+EDA+sulfo SMCC+nucleic acid product 34 is formed.

The attachment reaction of step S3 can in particular be carried out statically, that is to say without flow of a nucleic acid solution, or quasi-statically, that is to say with a flow almost zero, for example a flow greater than or equal to 0.01 μL/s and less than or equal to 0.05 μL/s.

During step S3, several nucleic acids can each be attached to a sulfo SMCC molecule 26 attached to the platinum electrode 20 via the EDA molecule 22. The self-assembled monolayer of EDA molecules supplemented with the sulfo SMCC molecules previously mentioned is then supplemented with nucleic acids.

A stabilization step S4 can optionally be implemented after step S3. During this step S4, the electrode is placed in physiological solution for a period comprised between 20 minutes and 40 minutes, ideally 30 minutes.

At the end of step S3 and, where appropriate, step S4, a step of characterizing the Platinum+EDA+sulfo SMCC+nucleic acid electrode 34 can be implemented, using one of the measurements previously described: cyclic voltammetry and/or chronoamperometry and/or PEIS.

The method for attaching a nucleic acid to a platinum electrode as presented uses the attachment of the “linker” to the platinum electrode which then allows to attach the nucleic acid via a thiol function.

The use of the thiol function to directly attach the nucleic acid to the platinum electrode is not satisfactory, the affinity being low between platinum and this function.

All the steps of the method described here are carried out in aqueous solution, which allows to work with nucleic acids and attach them to the electrode. In particular, no organic solvent is used, such a solvent being able to degrade the nucleic acid and prevent its attachment to the platinum electrode.

### Platinum Electrode for Detecting a Target Nucleic Acid

For the detection of a target nucleic acid, it is possible to use a platinum electrode to which is attached a strand of probe nucleic acid complementary to the target nucleic acid.

For this purpose, the object of the invention is also a platinum electrode which comprises a molecule of ethylenediamine 22 attached to the electrode, a sulfo

SMCC molecule 26 of 4-(N-Maleimidomethyl)cyclohexane-1-carboxylic acid 3-sulfo-N-hydroxysuccinimide ester attached to the ethylenediamine molecule 22, a probe nucleic acid 32 being attached to the sulfo SMCC molecule 26, the probe nucleic acid 32 comprising a thiol function, the probe nucleic acid 32 being complementary of the target nucleic acid 36.

Such an electrode can be produced by the nucleic acid attachment method presented previously in the case where the attached nucleic acid is the probe nucleic acid complementary to the target nucleic acid 36.

The platinum electrode may in particular be a micro-electrode.

A micro-electrode is defined here as an electrode whose dimensions are at least less than or equal to 1000 μm.

The attachment method described above can be implemented for different types of platinum electrode, and in particular for a micro-electrode.

The micro-electrode can be produced by photolithography.

In particular, platinum can be deposited on a glass substrate in the form of a track obtained after deposition and lithography steps in a clean room. The electrode is comprised on this platinum track.

The micro-electrode has a length in a first direction and a width in a second direction perpendicular to the first direction.

The dimensions of the working micro-electrode can for example be 30 μm in width and 300 μm in length.

For the electrode characterization steps described previously in connection with the attachment method, a counter electrode can be placed close to the platinum electrode. This counter electrode can be manufactured simultaneously with the platinum electrode by deposition on the glass substrate by lithography in a clean room.

The dimensions of the counter electrode are chosen to be larger than the dimensions of the working electrode, for example 2000 μm in width and 300 μm in length. Such dimensions then make the counter electrode a micro-electrode.

Such a micro-electrode can be placed at least partly in a micro-fluidic channel.

A micro-fluidic channel is a channel allowing the flow of fluids and having dimensions in a plane transverse to the general flow direction of the fluids which are greater than or equal to 100 μm and less than or equal to 500 μm.

The channel has, in a plane transverse to the general flow direction of the fluids, a width in the plane of the electrode and a height perpendicular to the plane of the electrode.

It is possible to implement all the steps of the method for attaching a nucleic acid on a micro-electrode placed at least partly in a micro-fluidic channel.

Preferably, the channel is placed on the working micro-electrode such that the flow direction is perpendicular to the length of the electrode. If the length of the electrode is greater than the width of the micro-fluidic channel, then the effective length of the electrode, that is to say the length seen by the liquid transported in the channel, is fixed by the width of the channel. For example with a channel width of 300 μm, the effective length of the working electrode is 300 μm for a width of 30 μm, the effective length of the counter electrode is 300 μm for a width of 2000 μm.

More precisely, the fact that all the steps of the attachment method are carried out in an aqueous solution allows to work in a micro-fluidic channel. In particular, no organic solvent is used, which would have prevented working in a micro-fluidic channel, the structure of the latter and its connections essential to its fluid supply being able to be damaged by an organic solvent.

It should be noted that working with a micro-fluidic channel allows to work in controllable flow regimes, in particular to control convection. This has the advantage of obtaining certain reactions more quickly (taking place in the thickness of the diffusion layer created on the surface of the electrode) by reducing the diffusion time towards the electrode. The nucleic acid targets, present in the liquid flowing through the channel, collide more quickly with the electrode surface to initiate hybridization reactions with the probe nucleic acids attached to the platinum electrode.

To place a micro-electrode at least partially in a micro-fluidic channel, the micro-fluidic channel can be manufactured in a PDMS resin, for example in the form of a groove open on the surface of a PDMS resin part. The groove extends in a direction of extension of the micro-fluidic channel.

In a plane perpendicular to this direction of extension, the groove can have a rectangular section. In this case and as the groove is open, the PDMS resin defines only three sides of the rectangle of the rectangular section.

The PDMS resin is then bonded to the substrate carrying the micro-electrode, for example a glass substrate, so that the substrate closes the micro-fluidic channel. In the case where the groove has a rectangular section, the micro-fluidic channel has a rectangular section of which three sides are formed by the PDMS resin and the last side is formed by the substrate.

The PDMS resin is adjusted in position such that the electrode track lies at least partially within the micro-fluidic channel.

Other polymer materials other than PDMS resin can be used for the micro-fluidic channel such as polymers: PMMA, PMP, PVDF, COC, PET; or block copolymers, etc.

Other manufacturing technologies can be used to manufacture the microchannel such as chemical etching on glass, molding, embossing, or 3-D printing.

The different solutions providing the reagents can be inserted into the micro-fluidic channel to carry out the different steps of the method described above.

For the electrode characterization steps, a counter electrode can be placed in the micro-fluidic channel close to the platinum electrode beforehand.

### Device for Detecting a Target Nucleic Acid

The platinum electrode for detecting a target nucleic acid presented above can in particular be comprised in a device for detecting a target nucleic acid, the device further comprising a counter electrode and an electrical measurement system electrically connected to the platinum electrode and the counter electrode.

The platinum electrode to which the probe nucleic acid is attached is hereinafter called the working electrode.

The counter electrode is placed sufficiently close to the working electrode so as to be able to impose a potential difference between these electrodes and measure a current between them.

For this purpose, the device comprises an electrical measurement system configured to impose a potential difference between the electrodes, measure this potential difference and measure a current between the electrodes.

The device is configured so that a solution to be analyzed can be simultaneously brought into contact with the working electrode and the counter electrode.

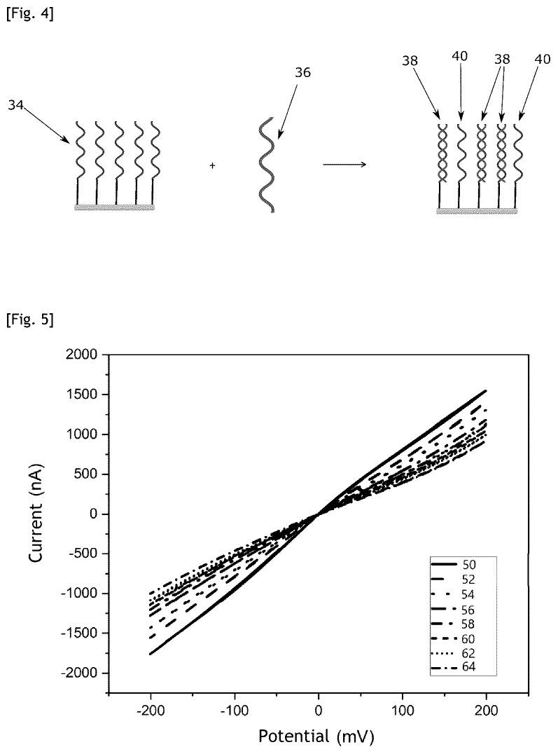

When the solution to be analyzed contains target nucleic acids, a hybridization reaction between the target nucleic acid and the probe nucleic acid occurs on the working electrode, as illustrated in FIG. 4.

The working electrode comprises a functionalized assembly 34 Platinum+EDA+sulfo SMCC+nucleic acid electrode as illustrated in FIG. 4. This functionalized assembly 34 comprises a probe nucleic acid which is complementary to a target nucleic acid 36.

The hybridization reaction occurs when the functionalized assembly 34 is brought into contact with a solution containing target nucleic acids 36. The target nucleic acid 36 and the probe nucleic acid 32 of the functionalized assembly 34 are hybridized.

In this way a hybrid 38 “probe nucleic acid 32+target nucleic acid 36” appears above the platinum electrode. The electrochemical properties of the electrode are modified by this hybridization so that the presence of target nucleic acid 36 can be detected.

When the platinum electrode comprises a plurality of probe nucleic acids, the presence of target nucleic acid 36 produces one or more hybrids 38 “probe nucleic acid 32+target nucleic acid 36” above the platinum electrode. Other probe nucleic acids which do not undergo hybridization remain unhybridized groups 40. The electrochemical properties of the electrode are modified by hybridization depending on the proportion between hybrids 38 and non-hybridized groups 40. These modifications allow to estimate the concentration of target nucleic acid in the solution to be analyzed.

The hybridization reaction is preferably carried out statically over a period greater than or equal to 15 minutes and less than or equal to 1 hour, preferably equal to 30 minutes.

The modification of the electrochemical properties of the electrode can be demonstrated by the electrical measurement system.

The graphs illustrated in FIG. 5 show such a modification.

The graphs were obtained experimentally using a platinum electrode for the detection of a target nucleic acid as described previously. More precisely, it is an electrode having a self-assembled monolayer of EDA molecules supplemented with sulfo-SMCC molecules and probe nucleic acids complementary to the target nucleic acid.

The electrode is used in a detection device as described, that is to say further comprising a counter electrode and an electrical measurement system electrically connected to the platinum electrode and to the counter electrode.

Different reference solutions having a concentration of the target nucleic acid equal respectively to 10-18, 10-16, 10-14, 10-12, 10-10, 108 and 106 mole/liter were used.

A neutral reference solution not comprising the target nucleic acid was also used. For each solution, the following electrochemical measurement is implemented:

the potential difference (VT-VCE) is varied between the potential VT of the working electrode and the potential VCE of the counter electrode and the electrical intensity between these electrodes is measured.

The potential difference (VT-VCE) is plotted on the abscissa axis of FIG. 5, the electrical intensity on the ordinate axis.

The potential difference (VT-VCE) is swept between-200 millivolts and +200 millivolts.

Initially, the electrodes are brought into contact with the reference solution to carry out the electrochemical measurement described above and illustrated by the curve 50.

The curve 50 appears as a quasi-linear curve in FIG. 5.

Then, other electrochemical measurements using the other reference solutions are implemented with the same device.

The respective curves 52, 54, 56, 58, 60, 62 and 64 represent the cases where the reference solution has a concentration of target nucleic acids equal respectively to 10-18, 10-16, 10-14, 10-12, 10-10, 10-8 and 10-6 mole/liter.

After each electrochemical measurement, the electrodes are rinsed with a physiological solution, that is to say a neutral reference solution.

A measurement with Fe(II)/Fe(III) is carried out to ensure that the surface of the working electrode has been modified on the one hand by the SAMs if necessary and on the other hand by the probe and target nucleic acids.

The electrodes are rinsed again with a physiological solution and then an electrochemical measurement is carried out with a new reference solution having a non-zero target nucleic acid concentration.

Care can be taken to carry out the measurements in increasing order of the nucleic acid concentrations of the reference solutions.

It is also possible to change the set of the two electrodes (working electrode and counter electrode) between each measurement.

The curves 52, 54, 56, 58, 60 and 62 also appear close to linear curves in FIG. 5, and the slope of this curve is increasingly low as the concentration of target nucleic acids increases. In other words, the electrochemical properties of the electrode are modified in the presence of target nucleic acids, the modification being even more significant as the concentration of target nucleic acids is high. A device for detecting a target nucleic acid as presented previously allows to detect the presence or absence of a nucleic acid in a solution brought into contact with its electrodes.

This experiment was carried out with probe DNA sequences to detect coding RNA targets (E, N, RdRp) and a negative control sequence. Table 1 mentions the different sequences used.

TABLE 1
Sequences used to produce a nucleic acid probe
on the working electrode. The DNA Probes are
modified by a thiol function
(—SH) in the 5′ position.

| | Corresponding |
|---|---|
| Probe name | DNA/RNA sequence |
| Probe DNA-Gene E | 5′-TCG-CTA-TTA-AGT-ATT-AAC- |
| | GTA-CCT-GT-3′ |
| Probe DNA-Gene N | 5′-GAT-TGC-GGG-TGC-CAA-TGT- |
| | G-3′ |
| Probe DNA-Gene RdRP | 5′-CCG-CCA-CAC-ATG-ACC-ATC- |
| | TCA-C-3′ |
| Target RNA-Gene E | 5′-ACA-GGU-ACG-UUA-AUA-CUU- |
| | AAU-AGC-GU-3′ |
| Target RNA-Gene N | 5′-CAC-AUU-GGC-ACC-CGC-AAU- |
| | C-3′ |
| Target RNA-Gene RdRP | 5′-GUG-AGA-UGG-UCA-UGU-GUG- |
| | GCG-G-3′ |
| DNA Target-poly(A) | 5′-AAA-AAA-AAA-AAA-AAA-AAA- |
| | AAA-AAA-AA-3′ |

Optionally, the device further comprises a micro-fluidic channel, the platinum electrode and the counter electrode being micro-electrodes, the platinum electrode and the counter electrode being placed at least partly in the micro-fluidic channel.

The device is configured so that in operation, fluids can flow through the micro-fluidic channel and come into contact with the working electrode and the counter electrode.

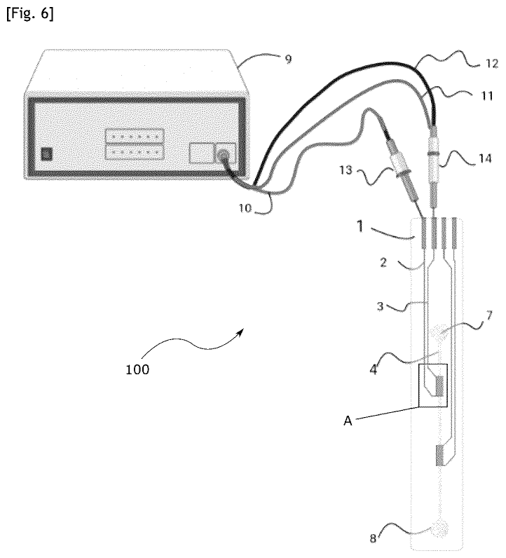

FIG. 6 schematically represents an example of a device 100 for detecting a target nucleic acid.

The device 100 comprises a glass substrate 1 on which platinum is deposited in the form of two metal tracks 2 and 3, including a working track 2 and a reference track 3. This deposition can in particular be carried out by deposition steps and clean room lithography as mentioned previously.

The working track 2 has a metallic termination called working electrode 5. It is to this working electrode 5 that the probe nucleic acid is attached.

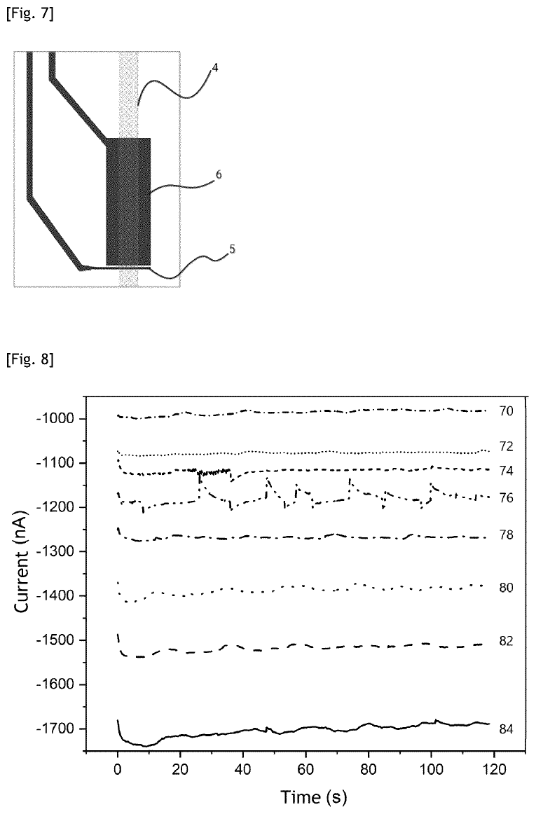

The reference track 3 has a metallic termination called counter electrode 6. The working electrode 5 and the counter electrode 6 are visible in FIG. 7 which is an enlargement of zone A of FIG. 6.

A micro-fluidic channel 4 of rectangular section is formed by bonding a PDMS resin to the glass substrate 1. The micro-fluidic channel 4 is located relative to the substrate so as to integrate part of the metal terminations of the metal tracks 2 and 3.

The dimensions of the micro-fluidic channel 4, the working electrode 5 and the counter electrode 6 are adjusted so that only part of the working electrode 5 and only part of the counter electrode 6 are comprised in the micro-fluidic channel.

In addition, the surface of the counter electrode 6 in the channel 4 is much greater than the surface of the working electrode 5 in the channel 4. This allows to use the counter electrode 6 not only as a counter electrode but also as a pseudo-reference.

The micro-fluidic channel 4 has an inlet 7 and an outlet 8. The fluids introduced through the inlet 7 pass through the micro-channel 4 to the outlet 8. During this crossing, the fluids come into contact with the working electrode 5 and the counter electrode 6. The fluids do not come into contact with the rest of the metal tracks 2 and 3.

The device 100 comprises an electrical measurement system configured to impose a potential difference between the electrodes, measure this potential difference and measure a current between the electrodes.

The electrical measurement system may in particular be made up of the following elements:
an instrument 9 of potentiostat type and constituting the potentiostat electrometer, the instrument 9 having three outlets, namely a working outlet 10, a reference outlet 11 and a counter electrode outlet 12,
a working connector 13 connecting the working outlet 10 to the metal track 2,
a reference connector 14 connecting the reference outlet 11 and the counter electrode outlet 12 to the reference track 3.

### Detection System Comprising a Plurality of Detection Devices

Another object of the invention is a target nucleic acid detection system comprising a plurality of nucleic acid detection devices as have just been presented.

The system comprises an inlet configured to receive a solution to be analyzed, the inlet connected to each platinum electrode of each device. In other words, a solution to be analyzed which is introduced into the inlet of the system moves through the system until it enters each device and comes into contact with the working electrode and the counter electrode.

The devices are preferably disposed in parallel, that is to say the inlet of the system is connected directly to each platinum electrode of each device. A solution to be analyzed that is introduced into the system inlet travels through the system until it simultaneously enters each device and contacts the working electrode and the counter electrode.

A detection system allows several measurements to be carried out simultaneously. For example, each detector can be configured to detect the same nucleic acid. The introduction of a solution to be analyzed allows to carry out the detection measurement or even measurement of the nucleic acid concentration several times. Such a detection system thus allows to obtain a measurement of greater precision.

FIG. 10 schematically represents an example of detection system 200, which comprises eight devices 42 for detection. Each detection device comprises a liquid inlet 47 and a fluid outlet 48. The inlets 47 can each be connected directly to the system inlet to obtain a parallel configuration. Each detection device here comprises two sets 50, 52 “working electrode and counter electrode”. Each electrode is connected to an electrical connection zone 46 which allows to electrically connect the electrode to its electrical measurement system.

The detection system may advantageously comprise two devices configured to detect two different target nucleic acids. In this case, the detection system allows to simultaneously carry out measurements concerning different nucleic acids.

In particular, each device in the system may be configured to detect a target nucleic acid different from the nucleic acids that the other devices are configured to detect. In this case, the detection system allows to simultaneously carry out measurements concerning as many different nucleic acids as there are devices in the system.

### Method for Detecting a Target Nucleic Acid

Another object of the invention is a method for detecting a target nucleic acid in a solution to be analyzed comprising the following steps:
a step E1 of providing a working electrode, the working electrode being a platinum electrode for the detection of a nucleic acid as presented previously,
a step E2 of providing a counter electrode,
a step E3 of electrically energizing the working electrode and the counter electrode so as to maintain a working voltage between them,
a step E4 of bringing the working electrode and the counter electrode into contact with the solution to be analyzed,
a step E5 of determining a measured intensity of an electric current between the working electrode and the counter electrode,
a step E6 of determining the presence of a target nucleic acid in the solution to be analyzed based on the measured intensity.

Steps E1 and E2 of the detection method can be implemented by providing a device for detecting a target nucleic acid as described previously.

Step E3 can be implemented using an electrical measurement system configured to impose a potential difference between the electrodes, measure this potential difference and measure a current between the electrodes.

Typically, the potential difference (VT-VCE) between the potential VT of the working electrode and the potential VCE of the counter electrode can be chosen greater than or equal to −250 millivolts and less than or equal to +250 millivolts, from preferably greater than or equal to −250 millivolts and less than or equal to −150 millivolts, preferably greater than or equal to −220 millivolts and less than or equal to −180 millivolts, preferably equal to −200 millivolts.

Step E4 of bringing the working electrode and the counter electrode into contact with the solution to be analyzed can be carried out statically or dynamically.

In static mode, a single block and fixed quantity of solution is brought into contact with the electrodes so as to bring the electrodes into electrical contact with the solution.

Dynamically, a continuous flow of solution to be analyzed passes into contact with the electrodes so as to bring the electrodes into electrical contact through the flow of solution. This dynamic implementation can in particular be carried out by a device for detecting a nucleic acid in the option where it comprises a micro-fluidic channel. A flow of solution to be analyzed can travel through the channel and come into contact with the electrodes. Typically, a solution flow can be generated in a micro-fluidic channel with a chosen value greater than or equal to 0.1 μL/second and less than or equal to 1 μL/second, preferably equal to 0.5 μL/second.

In all cases, contact with the solution to be analyzed is adapted to produce electrical contact between the electrodes by the solution to be analyzed.

The solution to be analyzed is an aqueous solution and can in particular be a physiological solution.

During this step E4 and when the solution to be analyzed contains target nucleic acids, a hybridization reaction between the target nucleic acid and the probe nucleic acid occurs on the working electrode, as presented previously in relation to FIG. 4.

The electrochemical properties of the electrode are modified by this hybridization so that the presence of target nucleic acid in the solution to be analyzed can be detected.

Step E5 takes place when the working electrode and the counter electrode are brought into contact with the solution to be analyzed so as to produce an electrical contact between the electrodes by the solution to be analyzed.

Step E5 of determining a measured intensity of an electric current between the working electrode and the counter electrode can be implemented using the electrical measurement system.

Step E5 can in particular be implemented by a chronoamperometry measurement.

FIG. 8 illustrates an example of chronoamperometry measurement.

The graphs illustrated in FIG. 8 were obtained experimentally for different reference solutions, by setting the potential difference (VT-VCE) between the potential VT of the working electrode and the potential VCE of the counter electrode at −200 millivolts and measuring the electrical intensity between these electrodes over time.

The electrical intensity between the electrodes is plotted on the ordinate axis, the abscissa axis corresponding to time.

Curve 84 represents the case where the solution to be analyzed is a reference solution which does not comprise nucleic acids.

The measurement of curve 84 is carried out before the others.

The respective curves 82, 80, 78, 76, 74, 72 and 70 represent the cases where the reference solution has a concentration of target nucleic acids equal respectively to 10-18, 10-16, 10-14, 10-12, 10-10, 108 and 106 mole/liter. After each electrochemical measurement, the electrodes are rinsed with a physiological solution, that is to say a neutral reference solution. Care can be taken to carry out the measurements in increasing order of the nucleic acid concentrations of the reference solutions.

FIG. 8 shows that the average value over time of the measured intensity allows to differentiate the different concentrations of target nucleic acids: the higher the average value, the greater the concentration.

Step E6 of determining the presence of target nucleic acid in the solution to be analyzed can be carried out by comparing the measured intensity to a reference intensity or probe intensity measured previously for a reference solution containing no target nucleic acid. If the measured intensity is significantly different from the probe intensity, then the target nucleic acid is present in the solution to be analyzed. A significant difference is a difference that exceeds a certain threshold. This threshold can be evaluated from a measurement statistic of this type allowing to evaluate the standard deviation and the background noise. The threshold can for example be chosen equal to one standard deviation or several standard deviations, for example three standard deviations.

Optionally, the method further comprises a calibration step comprising the following sub-steps:
a first sub-step SE1 of supplying two reference solutions having different concentrations of target nucleic acid,
a second sub-step SE2 of measuring for each reference solution a reference intensity between the working electrode and the counter electrode, the electrodes being brought into contact with the reference solution, the electrodes being electrically energized so as to maintain the working voltage between them,
a third sub-step SE3 of determining a correspondence of current intensities between the working electrode and the counter electrode accordingly and of target nucleic acid concentrations in a solution to be analyzed, the method comprising a step of determining a target nucleic acid concentration in the solution to be analyzed from the measured intensity using the correspondence.

During the first sub-step SE1 two reference solutions having different and known concentrations of target nucleic acid are provided. Preferably a number of reference solutions greater than two presenting are provided: the greater this number, the better the calibration.

For example, the reference solutions may have target nucleic acid concentrations equal to 10-18, 10-16, 10-14, 10-12, 10-10, 10-8 and 106 mole/liter.

During the second sub-step SE2 each reference solution is successively used to bring the working electrode and the counter electrode into contact so as to establish electrical contact between them.

The electrodes are energized so as to maintain the working voltage between them, for example −200 millivolts.

As for step E5 of the target nucleic acid detection method, an intensity is measured between the electrodes. This intensity is called reference intensity since it is determined for one of the reference solutions whose nucleic acid concentration is known.

The determination can in particular involve the calculation of an average intensity over time obtained by chronoamperometry.

FIG. 8 previously described corresponds to the determination of eight reference intensities, each being associated with a known nucleic acid concentration.

During the third sub-step SE3, a correspondence between current intensities and nucleic acid concentrations is determined.

This correspondence allows to associate, at least over a certain range of intensities and a certain range of concentrations, at a current intensity a nucleic acid concentration and conversely at a nucleic acid concentration a current intensity.

This correspondence can be determined for example by carrying out a linear regression of the reference intensities as a function of the nucleic acid concentration.

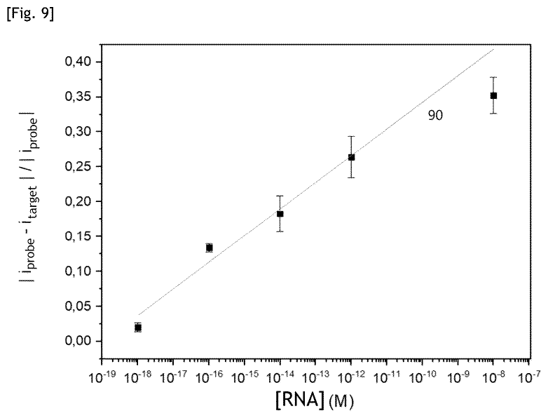

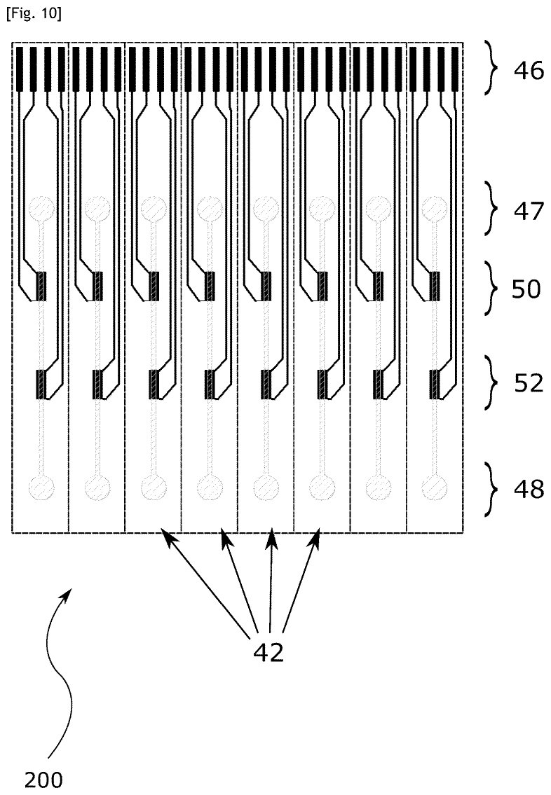

FIG. 9 illustrates another possible correspondence in which a relative intensity is determined before performing the linear regression.

This relative intensity is constructed from a probe intensity defined as the reference intensity for the reference solution not containing nucleic acids. Relative intensity is defined as the absolute value of the ratio of the difference between a reference intensity and the probe intensity to the probe intensity. The experimental values from the experiment relating to FIG. 8 are used to construct the points in FIG. 9.

A linear regression of these points is then carried out to obtain an adjustment curve 90.

The adjustment curve which models the relative intensity as a function of nucleic acid concentration constitutes a correspondence as sought.

The calibration step is completed when the correspondence between current intensities and nucleic acid concentrations is obtained.

The nucleic acid detection method can be refined to provide an estimate of nucleic acid concentration through correspondence.

The intensity measured for the solution to be analyzed whose nucleic acid concentration is unknown constitutes the inlet value in the correspondence. The correspondence allows this inlet value to be associated with an outlet value which is a nucleic acid concentration. This concentration then constitutes an estimate of the concentration of nucleic acids in the solution to be analyzed.

The detection method as described above can in particular be implemented when the target nucleic acid is a nucleic acid fragment of a pathogen, for example an RNA nucleic acid fragment encoding a coronavirus. Such a method can be useful in the field of rapid diagnosis for emergency biology. The method described allows to detect these fragments at trace levels, which is impossible using electrochemical technologies based on the use of a gold electrode.

In addition, it is possible with the method described to obtain quantitative and absolute measurements without resorting to a PCR technique-polymerase chain reaction-which involves reverse transcription-RT-of RNA into DNA, and amplification of the DNA strands allowing them to be brought to a detectable threshold.

1. A method for attaching a nucleic acid to a platinum electrode, the method comprising the following steps:
a step S1 of attaching an ethylenediamine molecule to the platinum electrode, the step S1 comprising an electro-oxidation by cyclic voltammetry of a primary amine comprised in the ethylenediamine molecule,
a step S2 of attaching a sulfo SMCC molecule of 4-(N-Maleimidomethyl)cyclohexane-1-carboxylic acid 3-sulfo-N-hydroxysuccinimide ester to the ethylenediamine molecule,
a step S3 of attaching the nucleic acid 32 to the sulfo SMCC molecule, the nucleic acid being previously modified so as to comprise a thiol function, the electrode being in contact with an aqueous solution during steps S1, S2 and S3, the aqueous solution being a solution in which the solvent is water.

2. The method according to claim 1 wherein the aqueous solution is a physiological solution.

3. The method according to claim 1 comprising a step of introducing the electrode at least partly in a micro-fluidic channel, the electrode being a micro-electrode.

4. The method according to claim 1 comprising after step S3 a step S4 of introducing the electrode in a physiological solution for a duration comprised between 20 minutes and 40 minutes, the solution having a sodium chloride (NaCl) concentration comprised between 0.4 molar and 0.6 molar.

5. A primary electrode for detecting a target nucleic acid, the primary electrode comprising:
a platinum electrode,
an ethylenediamine molecule attached to the platinum electrode,
a sulfo SMCC molecule of 4-(N-Maleimidomethyl)cyclohexane-1-carboxylic acid 3-sulfo-N-hydroxysuccinimide ester attached to a primary amine of the ethylenediamine molecule, and
a probe nucleic acid attached to the sulfo SMCC molecule,
the probe nucleic acid comprising a thiol function, the probe nucleic acid being complementary to the target nucleic acid.

6. A device for detecting a target nucleic acid comprising:
a primary electrode according to claim 5,
a counter electrode, and
an electrical measurement system electrically connected to the primary electrode and to the counter electrode.

7. The device according to claim 6, the device further comprising a micro-fluidic channel, the primary electrode being a micro-electrode, the primary electrode and the counter electrode being placed at least partly in the micro-fluidic channel.

8. A system for detecting target nucleic acids comprising a plurality of devices according to claim 6, the system comprising an inlet configured to receive a solution, the inlet being connected to each primary electrode of each device.

9. The detection system according to claim 8, two of the plurality of devices being configured to detect two different target nucleic acids.

10. A method for detecting a target nucleic acid in a solution, the method comprising the following steps:
a step E1 of providing a primary electrode according to claim 5,
a step E2 of providing a counter electrode,
a step E3 of setting an electrical voltage between the primary electrode and the counter electrode,
a step E4 of bringing the primary electrode into contact with the solution and bringing the counter electrode into contact with the solution,
a step E5 of measuring an electrical intensity between the primary electrode and the counter electrode,
a step E6 of determining the presence of a target nucleic acid in the solution based on the electrical intensity.

11. The method according to claim 10 further comprising a calibration step comprising the following sub-steps:
a first sub-step SE1 of supplying two reference solutions having different concentrations of the target nucleic acid,
a second sub-step SE2 of measuring for each reference solution a reference electrical intensity between the primary electrode and the counter electrode, the primary electrode being in contact with the reference solution, the counter electrode being in contact with the reference solution, an electrical voltage between the primary electrode and the counter electrode being set,
a third sub-step SE3 of determining a correspondence of a reference electrical intensity as a function of a reference target nucleic acid concentration, the reference electrical intensity being measured between the working electrode and the counter electrode both dipped in a reference solution presenting the reference target nucleic acid concentration,
the method comprising a step of determining a target nucleic acid concentration in the solution based on the electrical intensity and the correspondence.

12. The method according to claim 10, wherein the target nucleic acid is a nucleic acid fragment of a pathogen.


SEQUENCE LISTING (7 sequences)

SEQ ID NO: 1 - DNA, 26 bp, synthetic construct
  misc_feature 1..26: note = ADN sonde - gene E
  source 1..26: mol_type = other DNA
tcgctattaagtattaacgtacctgt

SEQ ID NO: 2 - DNA, 19 bp, synthetic construct
  misc_feature 1..19: note = ADN sonde - gene N
  source 1..19: mol_type = other DNA
gattgcgggtgccaatgtg

SEQ ID NO: 3 - DNA, 22 bp, synthetic construct
  misc_feature 1..22: note = ADN sonde - gene RdRP
  source 1..22: mol_type = other DNA
ccgccacacatgaccatctcac

SEQ ID NO: 4 - RNA, 26 nt, synthetic construct
  misc_feature 1..26: note = ARN cible - gene E
  source 1..26: mol_type = other RNA
acaggtacgttaatacttaatagcgt

SEQ ID NO: 5 - RNA, 19 nt, synthetic construct
  misc_feature 1..19: note = ARN cible - gene N
  source 1..19: mol_type = other RNA
cacattggcacccgcaatc

SEQ ID NO: 6 - RNA, 22 nt, synthetic construct
  misc_feature 1..22: note = ARN cible - gene RdRP
  source 1..22: mol_type = other RNA
gtgagatggtcatgtgtggcgg

SEQ ID NO: 7 - DNA, 26 bp, synthetic construct
  misc_feature 1..26: note = ADN cible - poly(A)
  source 1..26: mol_type = other DNA
aaaaaaaaaaaaaaaaaaaaaaaaaa